(19) United States
(12) Patent Application Publication
(10) Pub. No.: US 2010/0080856 A1
(43) Pub. Date: Apr. 1, 2010

(54) Method and composition for postoperative wound healing

(76) Inventor: Peter D. Costantino, Armonk, NY (US)

Correspondence Address: Michael G. Petit, 134 Skyline Drive, Coram, NY, 11727, US

(21) Appl. No.: 12/284,966

(22) Filed: Sep. 26, 2008

(51) Int. Cl. A61K 33/34; C07C 229/02; A61K 31/225; A61K 31/375; A61K 31/593; A61K 31/51; A61K 31/525; A61K 33/04; A61P 17/02; A61K 33/32; A61K 31/455; A61K 31/44; A61K 33/06; A61K 33/26; A61K 33/30

(52) U.S. Cl. 424/630; 560/171; 514/547; 514/474; 514/167; 514/276; 514/251; 514/356; 514/345; 424/682; 424/646; 424/641; 424/639; 424/655; 424/702

(57) ABSTRACT

A composition and a method for administering the composition to a patient in order to accelerate wound healing, particularly postoperative wound healing. The composition, which essentially comprises ornithine α-ketoglutarate, is water-soluble and formulated for oral administration in single-dose packets. The method for using the composition for promoting wound healing includes the step of administering the composition to a patient preoperatively as well as-postoperatively in accordance with a prescribed protocol. The composition may be customized to include one or more additional agents that are effective for promoting nutritional health and/or treating a particular medical problem. The composition may also be taken daily by athletes to minimize the healing time of closed wounds such as muscle or tendon tears that occur during participation in sports, even when the symptoms of such injury are subclinical.

### BACKGROUND OF THE INVENTION

1. Field of the Invention

The present invention relates to a water soluble composition comprising ornithine α-ketoglutarate and, more particularly, to a method for using the composition to facilitate postoperative wound healing and as a dietary supplement for athletes to accelerate healing of injured tissue.

2. Prior Art

When an individual is wounded, a set of complex biochemical events takes place in a closely orchestrated cascade to repair the damage. Although these events overlap in time, they may be separated into distinct phases: (a) the inflammatory phase; (b) the proliferative phase; and (c) the remodeling phase. During the inflammatory phase, bacteria and debris within the wound are phagocytized and removed, and factors are released that cause the migration and division of cells involved in the proliferative phase. The proliferative phase is characterized, inter alia, by angiogenesis, collagen deposition, granulation tissue formation, epithelialization, and wound contraction. During angiogenesis, new blood vessels are formed from endothelial cells. In granulation tissue formation, fibroblasts form an extracellular matrix by excreting collagen and fibronectin.

During epithelialization, epithelial cells migrate across the wound bed to form a layer substantially coextensive with the wound bed. In contraction, the wound is made smaller by the action of myofibroblasts, which establish a grip on the wound edges and contract themselves using a mechanism similar to smooth muscle cell contraction. In the maturation and remodeling phase, collagen is realigned along tension lines and cells that are no longer needed are removed by apoptosis. The entire wound healing process is complex and susceptible to interruption or failure leading to non-healing or slow-healing wounds. Endogenous chronic factors which may impair wound healing include diabetes, vascular disease, old-age and-infection.

In addition to the aforesaid chronic illnesses that impair wound healing, the general nutritional condition of the patient is also a factor in the rate of wound healing. During each step of the wound healing process, the biosynthesis and secretion of essential factors must occur. Dietary intervention, whether in the form of general nutritional support or as a single nutrient supplementation such as, for example, an essential vitamin or a proteolytic enzyme, may improve or accelerate the wound healing process. Oral nutritional supplements have been shown to accelerate skin wound healing. (Plast. Reconstr. Surg. 114: 237, 2004). Coudray-Lucas et al. (Crit Care Med. 2000, Vol 28, No. 6, 1772-1776) report that the oral administration of ornithine α-ketoglutarate (OKG) shortens the wound healing time in severe burn patients. Maintaining an optimum, wound healing environment is essential for trauma patients inasmuch as the wound healing process is activated immediately after the wound formation. Unlike fat and carbohydrates, there is no storage of protein in the body. Every molecule of protein is serving a vital function. When protein is catabolized to glycogen, vital functions such as wound healing and immunity are compromised.

One of the earliest effects of protein malnutrition is the depression of the formation (in the liver) of serum transport proteins. As serum protein levels decrease, the amino acid pool decreases due to the above-mentioned fact that there is no protein storage in the human body. There must be adequate amounts of transport proteins and adequate circulation and tissue perfusion in and around the wound to allow healing to progress and promote immunity to invading bacteria. Adequate amounts of serum proteins are essential, both for their transportation function as well as for their maintenance of osmotic pressure to avoid tissue edema. Inadequate availability of amino acid substrate impairs the rate and quality of tissue synthesis during wound healing. Initially, the wound healing process takes priority for utilization of protein substrates. Any loss of a patient's lean body mass greater than 15% of total lean body mass will result in a diversion of protein substrate away from the wound and back toward restoration of lean mass. The greater the net loss of body protein before, during and after wound formation, the greater the impairment to healing.

The unavailability of key amino acids such as glutamine, arginine, proline and other micronutrients either at, or subsequent to, the time a wound is formed will impair the rate of wound healing. In the case of elective surgery, there is an opportunity for the health care practitioner to preoperatively modulate the supply and availability of such key amino acids and nutrient compositions in a patient prior to the actual formation of a wound as well as subsequent to the wounding event. There is a continuing need for compositions that may be administered to a patient, and a method or protocol for administering the composition to the patient that will optimize the general nutrition of the patient in preparation for healing surgically induced wounds.

During vigorous physical training and sport performance, athletes incur injuries to soft tissue such as muscle, tendons or ligaments wherein the symptoms of the injury may be either clinically apparent or subclinical. Such injury may be viewed as a “closed wound.” Clinical symptoms that accompany such closed wounds are usually treated by icing and/or administration of an antiinflammatory agent. Wounds that present no clinical symptoms go untreated. While such symptomatic treatment may ease the athlete's symptoms, it does little to promote tissue healing. The present invention is an orally acceptable composition that when taken daily optimizes serum concentrations of factors critical to the wound healing process in closed wounds.

### SUMMARY

The present invention is directed to orally acceptable micronutrient compositions comprising ornithine α-ketoglutarate (“OKG”) and a method for using the compositions to assist the wound-healing process in the body following surgery. All compositions of the present invention are essentially a water soluble powder packaged in a unit dose comprising about 10 grams of ornithine α-ketoglutarate. The composition is intended to be orally ingested by a patient two times daily beginning at least 2 days prior to the elective surgery.

In a preferred embodiment of the micronutrient composition a unit dose comprises 10 grams of OKG, and further includes Vitamin A, Vitamin C, Vitamin D3, Vitamin-K, Thiamin, Vitamin-B-₁₂, Riboflavin, Niacin, Pantothenic acid, Pyridoxine, Folate, Biotin, Calcium, Magnesium, Iron, Zinc, Copper, Manganese, Chromium, Selenium, Potassium, Boron, Choline, N-Acetyl Cysteine, L-Glutamine, Glycine propionylcamitine, Medium chain triglycerides, bromelain, Carnitine fumarate and a blend of the levorotatory form of the amino acids: leucine, lysine, threonine, tyrosine, histadine, isoleucine, valine and methionine. In addition to the above active ingredients, the composition may further comprise ribose, dextrose and whey protein as flavoring agents, coloring agents and thixotropic agents such as cellulose and xanthan gums.

The present invention is further directed to a method for using the compositions to accelerate the wound-healing process in athletes that are at risk of injury during the performance of a sport-related activity. The symptoms of such injury may be clinically apparent or subclinical. The method includes the daily self-administration of the micronutrient composition comprising about 20 grams of ornithine α-ketoglutarate, the daily dosing to begin about a month prior to engaging in such activity and to continue until about a month after such vigorous activity terminates.

The features of the invention believed to be novel are set forth with particularity in the appended claims. However the invention itself, both as to organization and method of operation, together with further objects and advantages thereof may be best understood by reference to the following description.

### DESCRIPTION OF THE PREFERRED EMBODIMENTS

Surprisingly, while ornithine α-ketoglutarate (OKG) has been used reactively (i.e., after a wound is formed) to promote wound healing such as, for example, in burn patients, it has not been used proactively as part of a daily nutrient supplement regimen to the patient with a nutrient pool that will accelerate post-operative wound healing. Such a preoperative dosing regimen may significantly reduce the healing time for patients undergoing elective surgical procedures such as, for example, facelifts or liposuction.

Ornithine α-ketoglutarate is a salt comprised of 2 molecules of ornithine and 1 molecule of α-ketoglutarate. Its administration improves nutritional status in chronically malnourished (e.g., elderly) and acutely malnourished patients (especially burn and trauma patients). There is evidence that OKG activity is not the simple addition of the effects of ornithine and α-ketoglutarate, because the presence of both moieties is required to induce the generation of key metabolites such as glutamine, proline, and arginine, whereas this does not occur when one or the other is given separately.

Ornithine α-ketoglutarate (OKG) is a nitrogenous compound that stimulates the secretion of insulin and growth hormone as well as the synthesis of the amino acids glutamine, arginine and proline. It also supports the increased division of fibroblasts and can boost immunity. OKG has proven to be effective in reducing both wound healing time and infections in cases of reconstructive surgery and severe burns when administered to patients through a feeding tube. There are several possible explanations for this. The rate of collagen synthesis, for instance, depends on the concentration of the amino acid proline present, and OKG is a precursor to proline. Once collagen is synthesized, its deposition in the wound is dependent on arginine, which is also derived from OKG. The ability of OKG to prevent excessive breakdown of muscle proteins in burn patients may also account for the improved healing times in such patients.

For years, ornithine α-ketoglutarate activity has been associated with its ability to induce the secretion of anabolic hormones, such as insulin and growth hormone, and to increase glutamine and polyamine synthesis. Recent studies using chemical inhibitors of nitric oxide synthase (NOS) suggest that nitric oxide derived from arginine could be partly involved in OKG activity.

Theoretically, α-ketoglutarate is a precursor of glutamine, a fact that may be of importance given the key regulatory properties of this amino acid. Although the literature suggests that glutamine synthesis accounts only for a marginal part of the disposal of exogenously supplied α-ketoglutarate, administered α-ketoglutarate has a potent conserving effect on endogenous glutamine pools. When α-ketoglutarate is supplied as an ornithine salt, a synergistic effect of the two parts of the molecule increases the synthesis of glutamine or the conservation of endogenous glutamine pools. In addition, α-ketoglutarate, in combination with ornithine, dramatically increases the synthesis of arginine, proline and polyamines, which also play key roles in metabolic sequellae to wounding. The administration of about 10 grams of ornithine α-ketoglutarate in an orally acceptable fluid twice daily, both pre and postoperatively, improves anabolic/anticatabolic actions on protein metabolism and accelerates wound healing. It is a feature of the present invention that a unit dose of the composition (which contains about 10 grams of ornithine α-ketoglutarate) be orally administered to the patient twice daily beginning at least 2 days prior to surgery and ending about 1-2 weeks after surgery.

In addition to the administration of ornithine α-ketoglutarate to a patient before and following surgery, one or more additional nutrients may be included within the composition of the present invention.

Additional Nutrients

Vitamin A (as retinyl palmitate) refers to a family of fat-soluble vitamins most known for its role in maintaining vision. However, vitamin A also helps to fight infections in the human body. It may enhance wound healing by increasing the number of certain types of white blood cells called monocytes and macrophages in the wound. It restores the initial phase of wound healing, also known as the inflammatory phase, which is characterized by redness, heat, swelling, pain, and loss of function at the wound site. The wound healing ability of vitamin A is especially important in patients who are taking steroid medications for long-term therapy. The use of these steroids can negatively affect the wound healing process, but taking vitamin A can counteract this problem. Research has shown improvement in the breaking strength of wounds, or the maximum stress the wound can take until it is disrupted, when vitamin A is given in addition to steroid medication.

Vitamin C ((as Ester-C) is a cofactor in the conversion of the amino acid proline to hydroxyproline, which is an important step in the production of collagen. Vitamin C also stimulates the growth and division of fibroblasts. Vitamin C supplementation has been proven to enhance wound healing in deficient patients who are also more likely to have wound infections that may happen because of impaired collagen synthesis. Some evidence indicates that grape seed extract helps vitamin C enter cells to strengthen the cell membranes and protect the cells from oxidative damage, or damage caused by reactions that are promoted by oxygen and peroxides. Vitamin C supplementation in healthy individuals remains a much debated issue.

Vitamin D3 (as cholecalciferol) is a natural form of vitamin D made in the body and also commonly found in fish, egg yolks, and fish-liver oil. It may help the wound healing process since it acts as a powerful anti-inflammatory agent. Production of vitamin D3, which takes place in the skin, is influenced by various factors including the extent of the presence of melanin, the pigment that gives skin its color and sun protection. The relationship between increased pigmentation and decreased presence of vitamin D3 in the skin may account for the development of undesirable scarring during the wound healing process in dark-skinned patients. Animal studies have shown vitamin D3 to produce increases in wound breaking strength and promote epithehization, the movement of skin cells to the wound site, implying a biological role of vitamin D3 in human wound repair as well.

Vitamin K (as phytonadione) is a vitamin found in green leafy vegetables such as spinach, sprouts, broccoli and cauliflower (and, to a lesser degree, in liver, lean meat, cow's milk, egg yolk and whole wheat products). It is required to make several of the factors involved in the blood clotting sequence such as prothrombin; Factors VIII, IX, and X; and proteins C, S, and Z. Vitamin K is also essential for the synthesis of other vitamin K dependent proteins found in bones, plasma and kidneys. However, its role in blood clotting is what makes vitamin K essential to the wound healing process. Without vitamin K to control bleeding at the wound site, no surgical scar could form or heal properly.

The B vitamins (Vitamin B₁—Thiamin (as thiamin HCL), Vitamin B₂—(as methylcobalamin), Riboflavin, Vitamin B₃—Niacin (as niacinamide), Vitamin B₅—Pantothenic acid (as D-calcium pantothenate) and Vitamin B₆—Pyridoxine (as pyridoxine HCL)) act as cofactors or helpers for a number of enzymes that are required for over 100 protein, carbohydrate and lipid metabolic reactions. As a result, a deficiency in these vitamins would have a negative effect on the production of the energy and amino acids necessary for the wound healing process. In fact, studies on animals have shown that deficiencies in B vitamins such vitamins B, and B₅ lead to impairment in the rate and quality of wound healing. Vitamin B complex may also have an indirect role in wound healing through its influence on host resistance, the ability of the patient to fight off infection. As with all water-soluble vitamins, they should not be consumed on a regular basis since the body does not store them.

Methylcobalamin, a form of Vitamin B₁₂, acts as a coenzyme in the metabolism of nucleic acids and helps to form hemoglobin, an iron-containing pigment in red blood cells that helps transport oxygen from the lungs to the tissues in the body. Formation of hemoglobin is important for prevention of megaloblastic anemia, a type of anemia in which there are a lot of large immature red blood cells circulating in the blood. Methylcobalamin is commonly given to lower levels of homocysteine, an amino acid that can increase risk for heart disease and lead to complications in wound healing. Methylcobalamin is also involved in repairing nerve cells, specifically the myelin sheath that insulates nerve fibers and allows damaged neurons to regenerate. This, in addition to its ability to lower homocysteine levels, makes methylcobalamin an important vitamin for the wound healing process.

Folate (as metafolin (methyltetrahydrofolate)) is a form of the vitamin known as folic acid. Folic acid plays a key role as a coenzyme, or enzyme helper, in metabolic reactions of amino acids and nucleic acids, which are the building blocks of proteins and DNA, in that order. A folic acid deficiency can therefore lead to a decrease in cell division and the production of DNA. This can lead to anemia, a condition in which there is not enough red blood cells circulating in the blood; The toxicity risk of folic acid supplementation has been shown to be very low.

Biotin is a water-soluble B vitamin that acts as a cofactor in enzyme reactions involving carbon dioxide. Recent studies suggest that biotin is also necessary for processes on the genetic level in cells, most importantly DNA replication and gene expression. Biotin deficiency can cause a number of complications involving the skin, intestinal tract, and nervous system. Ingestion of raw egg whites is just one cause of biotin deficiency, since all available biotin in the body will be used up by binding very tightly to the protein avidin found in egg whites. Since it is an important micronutrient (a nutrient only needed in small amounts) for normal function, growth, and development of cells, biotin indirectly helps the wound healing process.

Calcium (as calcium citrate) is one of a number of cofactors involved in the production of collagen, a very tough fiber-like protein that gives skin elasticity and strength. It is mainly involved in the very first phase of wound repair involving the blood clotting process, also known as the hemostatic phase. Calcium is also required in later stages of healing, particularly for the migration or crawling of skin cells to the wound site for the purpose of covering the wound. Although it is not known exactly how the level of calcium is controlled in the wound site, experimental evidence has shown that calcium is definitely required for wound management.

Magnesium (as dimagnesium malate) acts as a cofactor in the synthesis of collagen and other proteins. It helps to stabilize the structure of adenosine triphosphate (ATP), which is responsible for powering many of the processes involved in collagen production. Thus, magnesium is essential for wound repair.

Iron (as ferrous glycinate) acts as a cofactor in collagen production. Its importance in wound healing is mostly in connection with the later stages of wound healing, the proliferative and remodeling phases. An iron deficiency is directly reflected at the wound site, since oxygen delivery to the wound site is directly proportional to the concentration of oxygen circulating in the blood. In cases of severe iron-deficiency anemia, oxygen will be conserved to be transported mainly to the vital organs, so that the wound will be further deprived of oxygen and the repair process at the wound bed will slow down. Iron is also required for the ability of the body to fight off infection of the wound.

Zinc (as zinc arginate) forms part of more enzymes and macromolecules than any other isolated nutrient. A deficiency in zinc affects basic cell division, differentiation, and growth processes, which are all important for wound healing. A number of studies in animal and human models indicate that zinc is most important during the later stages of tissue repair and regeneration, since it influences processes taking place in these later stages such as reepithelialization (the migration of skin cells to the wound site) and collagen deposition. Zinc has also been shown to help increase wound breaking strength, modulate integrins (proteins released from skin cells that promote wound healing), and activate the enzyme lysyl oxidase, which is responsible for the formation of the links between collagen fibers. However, zinc supplementation must be monitored very carefully since too much zinc in the plasma can lead to deficiencies in vitamin A and copper, which are both key in the wound healing process as well.

Copper (as copper lysinate) is a trace element in the body with a close relationship to wound healing. Copper, like zinc, is a required cofactor for the enzyme lysyl oxidase, which plays a role in the cross-linking of collagen and strengthening of connective tissue. Copper has recently been shown to be involved in the production of vascular endothelial growth factor (VEGF), a protein that promotes angiogenesis (the growth of new blood vessels). This property of copper would certainly be useful in the acceleration of the wound healing process. Copper-based medications such as ointments have also shown to promote wound healing in a number of animal studies.

Manganese (as in manganese amino acid chelate) is a trace or microelement that functions as a cofactor involved in collagen production. It is important in both the initial and final stages of the healing process. As with zinc, one particular study has shown the influence of manganese on the expression of integrin proteins that promote wound healing in certain skin cells called keratinocytes. It is rare for healthy individuals to be deficient in trace elements such as manganese. Still, in certain parts of the world, direct application of medicinal plants with a high concentration of manganese and other trace elements to the wound site is practiced to cut down on the time the body spends on displacing and transporting these elements needed for enzyme activity in the wound.

Chromium (as chromium amino acid chelate) is a lesser-known trace element, required in only small amounts in the body. It is involved as a cofactor for many fat, carbohydrate and protein metabolic reactions. For this reason, it may help in the wound healing process.

Selenium (as selenomethionine) is a potential antioxidant, which means that it can fight against reactive substances called free radicals that can damage cells, proteins, and DNA by changing their chemical structure. Critically ill patients in particular have lower levels of selenium, and so early administration of selenium would help to prevent or correct free radical damage that may occur as a result of sepsis, the body's response to a serious infection. This is especially true in patients with burn injuries. However, there is little or debatable evidence for the use of selenium as a supplement for wound healing in healthy patients.

Potassium is required for the regulation of muscle contraction and nerve impulses. However, potassium also plays a role in wound healing. Part of the wound healing process involves the increased growth of fibroblasts, or connective-tissue cells that produce proteins like collagen to help rebuild new tissue called the extracellular matrix (ECM). Potassium has been shown to increase the number of fibroblasts that synthesize DNA, which indicates that these cells are actively growing and dividing.

Boron, in the form of calcium fructoborate, has been used as a nutritional supplement to promote healthy bones, joints and prostate and in the regulation of steroid-hormone levels.

L-Glutamine is the most abundant amino acid, both in the body's circulation and in individual cells. It plays an indirect role in wound healing. It is a source of fuel for rapidly dividing cells such as fibroblasts at the wound site. Glutamine has also been reported to enhance the immune response by influencing the action of immune cells, especially certain white blood cells called neutrophils. Studies suggest that glutamine may help in cases of severe infection and trauma as well as wound healing in severely burned patients. Glutamine may also be given as a dietary supplement to improve nitrogen balance after elective surgery, or, in other words, to make sure that the body's protein requirements for physiological processes are being met.

Choline in the form of alpha-glycerylphosphorylcholine (αGPC) is a nutrient derived from soy phospholipids, one of these phospholipids being lecithin. αGPC supports healthy levels of acetylcholine & phosphatidylcholine in the brain, somatostatin and GH while helping to promote fat burning, mental focus, cognitive function, strength, response time, balance & coordination.

N-acetyl-cysteine (NAC) is the acetylated form of L-cysteine, which is more efficiently absorbed and used because it may protect the cells directly. NAC is an antioxidant and a free radical-scavenging agent that increases intracellular GSH, at the cellular level. NAC can act as a precursor for glutathione synthesis as well as a stimulator of the cytosolic enzymes involved in glutathione regeneration. NAC has been shown to protect against oxidative stress-induced neuronal death in cultured granule neurons.

Medium chain triglycerides (MCTs) passively diffuse from the GI tract to the portal system (longer fatty acids are absorbed into the lymphatic system) without requirement for modification like long chain fatty acids or very long chain fatty acids do. In addition MCTs do not require bile salts for digestion. Patients who have malnutrition or malabsorption syndromes are treated with MCTs because they do not require energy for absorption, utilization, or storage.

Bromelain is an extract derived from pineapple stem. It contains various enzymes that are involved in the immune response, minimization of swelling, or breakdown of proteins in order to form the wound matrix. Several studies have shown bromelain to accelerate the wound healing process. Bromelain supplementation prior to and following a surgical procedure was found to result in reduction of swelling, bruising, healing time, and pain as well as soft-tissue healing acceleration particularly in male boxers. To prevent destruction by stomach acid, bromelain can be administered in the form of tablets coated with a special substance that protects it from breakdown in the stomach.

Proteolytic enzymes (proteases) help digest the proteins in food. Although the human body produces these enzymes in the pancreas, certain foods also contain proteolytic enzymes. The primary use of proteolytic enzymes is as a digestive aid for people who have trouble digesting proteins. However, proteolytic enzymes may also be absorbed internally to some extent and may reduce pain and inflammation.

Glycine propionyl L-Carnitine (GPLC) is a propionyl ester of carnitine (PLC) that includes an additional glycine component to promote energy used by muscles, such as the heart. It assists with energy production by facilitating the transport of fatty acids into the mitochondria. It also assists in the removal of waste substances. GPLC helps compensate for the reduced oxygen availability during stress. It promotes proper metabolism of carbohydrates, helps reduce the buildup of lactic acid, and provides powerful support for peripheral arterial blood flow.

L-Carnitine (supplied as L-Carnitine fumarate) is a naturally occurring substance found in most cells of the body, particularly the brain, neural tissue, muscle, and heart. The body uses Carnitine to transport fatty acids to cell mitochondria to be used for energy. Because fat burning is important for muscular energy, carnitine deficiencies show up as decreased energy levels and weakness of the muscles.

Ribose is a carbohydrate, or sugar, used by all living cells and is an essential component in our body's energy production. Ribose has many important roles in physiology. It is used by the body to synthesize ATP and glycogen for energy, and is a necessary substrate for synthesis of nucleotides, which is part of the building blocks that form DNA and RNA molecules.

A unit dose of a composition in accordance with a most preferred embodiment of the present invention includes 10 grams of ornithine α-ketoglutarate, 12,500 IU Vitamin A (as retinyl palmitate), 500 mg Vitamin C (as Ester-C), 500 IU Vitamin D3 (as cholecalciferol), 40 mcg Vitamin K (as phytonadione), 4.5 mg Vitamin B₁—Thiamin (as thiamin HCL), 50 mcg Vitamin B₁₂—(as methylcobalamin), 5.1 mg Riboflavin, 60 mg Vitamin B₃—Niacin (as niacinamide), 30 mg Vitamin B₅—Pantothenic acid (as cholecalciferol) and 6 mg Vitamin B₆—Pyridoxine (as pyridoxine HCL), 400 mcg Folate (as metafolin), 150 mcg Biotin, 300 mg Calcium (as calcium citrate), 200 mg Magnesium (as dimagnesium malate), 300 mg Iron (as ferrous glycinate), 15 mg Zinc (as zinc arginate), 1 mg Copper (as copper lysinate), 2 mg Manganese (as manganese amino acid chelate), 120 mcg Chromium (as chromium amino acid chelate), 100 mcg Selenium (as selenomethionine), 500 mg Potassium (as potassium citrate), L-Glutamine, 200 mg Choline (as choline bitartrate), 5 g medium chain triglycerides, 500 mg bromelain and about 8 grams of a blend of the levorotatory form of the amino acids: leucine, lysine, threonine, tyrosine, histadine, isoleucine, valine and methionine. In addition, the composition may further include inactive ingredients including, but not limited to, dextrose, natural and artificial flavors, colorings and gums.

In accordance with the preferred method of accelerating wound healing of the present invention, about 2-10 days prior to elective surgery, a unit dose of the composition, which comprises inter alia 10 g ornithine α-ketoglutarate, is orally administered perioperatively to a surgical patient twice daily commencing at least two days prior to surgery and terminating about one week after surgery. The method enables the surgeon to proactively improve the nutrition of the patient prior to surgery to optimize the availability-of nutrients required-for wound healing. Most preferably, a unit dose of the composition comprises 10 g ornithine α-ketoglutarate, Vitamin A (as retinyl palmitate), Vitamin C (as Ester-C), Vitamin D3 (as cholecalciferol), Vitamin K (as phytonadione), Vitamin B₁—Thiamin (as thiamin HCL), Vitamin B₂—(as methylcobalamin), Riboflavin, Vitamin B₃—Niacin (as niacinamide), Vitamin B₅—Pantothenic acid (as cholecalciferol) and Vitamin B₆—Pyridoxine (as pyridoxine HCL), Folate (as metafolin), Biotin, Calcium (as calcium citrate), Magnesium (as dimagnesium malate), Iron (as ferrous glycinate), Zinc (as zinc arginate), Copper (as copper lysinate), Manganese, Chromium (as chromium amino acid chelate), Selenium (as selenomethionine), Potassium, Choline (as choline bitartrate), N-acetyl-cysteine, Medium chain triglycerides, a blend of the levorotatory form of the amino acids: leucine, lysine, threonine, tyrosine, histadine, isoleucine, valine and methionine; and bromelain.

Many athletes, particularly professional athletes playing on a team, suffer injury during practice and/or competition. The healing of such injuries may require substantial time wherein the athlete is unable to compete. Even minor injuries wherein the athlete's symptoms are subclinical, such as microtears and fractures, can lead to impaired performance. If the athlete's performance is impaired, or during periods of inactivity of a paid athlete, impaired team performance may have a negative financial impact on the team. It is a further object of the present invention to provide athletes with a composition and method for using the composition that, when taken daily, can substantially reduce the time required for wound healing.

In accordance with the aforesaid method of accelerating wound healing in injured athletes, about 1 month prior to incurring the risk of injury and daily thereafter, a unit dose of the composition is orally self-administered twice daily by the athlete. A unit dose of the composition essentially comprises 10 g ornithine α-ketoglutarate. Most preferably, a unit dose of the composition comprises 10 g ornithine α-ketoglutarate, Vitamin A (as retinyl palmitate), Vitamin C (as Ester-C), Vitamin D3 (as cholecalciferol), Vitamin K (as phytonadione), Vitamin B₁—Thiamin (as thiamin HCL), Vitamin B₂—(as methylcobalamin), Riboflavin, Vitamin B₃—Niacin (as niacinamide), Vitamin B₅—Pantothenic acid (as cholecalciferol) and Vitamin B₆—Pyridoxine (as pyridoxine HCL), Folate (as metafolin), Biotin, Calcium (as calcium citrate), Magnesium (as dimagnesium malate), Iron (as ferrous glycinate), Zinc (as zinc arginate), Copper (as copper lysinate), Manganese, Chromium (as chromium amino acid chelate), Selenium (as selenomethionine), Potassium, Choline, N-acetyl-cysteine, Medium chain triglycerides, a blend of the levorotatory form of the amino acids: leucine, lysine, threonine, tyrosine, histadine, isoleucine, valine and methionine; and bromelain.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What I claim is:

1. A micronutrient composition packaged in a unit dose wherein said unit dose comprises essentially about 10 grams ornithine α-ketoglutarate

2. The micronutrient composition of claim 1, wherein said unit dose further includes one or more micronutrients selected from the group consisting of: Vitamin A, Vitamin C, Vitamin D3; Vitamin K, Thiamin, Vitamin B₂, Riboflavin, Niacin, Pantothenic acid, Pyridoxine, Folate, Biotin, Calcium, Magnesium, Iron, Zinc, Copper, Manganese, Chromium, Selenium, Potassium, Choline, Medium chain triglycerides, bromelain and the levorotatory form of the amino acids leucine, lysine, threonine, tyrosine, histadine, isoleucine, valine and methionine.

3. The micronutrient composition of claim 1, wherein said unit dose is a water soluble powder.

4. A micronutrient composition packaged in a unit dose, said unit dose of said composition consisting essentially of about 10 grams of ornithine α-ketoglutarate, 12,500 IU Vitamin A (as retinyl palmitate), 500 mg Vitamin C (as Ester-C), 500 IU Vitamin D3 (as cholecalciferol), 40 mcg Vitamin K (as phytonadione), 4.5 mg Vitamin B₁—Thiamin (as thiamin HCL), 50 mcg Vitamin B₁₂—(as methylcobalamin), 5.1 mg Riboflavin, 60 mg Vitamin B₃—Niacin (as niacinamide), 30 mg Vitamin B₅—Pantothenic acid (as cholecalciferol) and 6 mg Vitamin B₆—Pyridoxine (as pyridoxine HCL), 400 mcg Folate (as metafolin), 150 mcg Biotin, 300 mg Calcium (as calcium citrate), 200 mg Magnesium (as dimagnesium malate), 300 mg Iron (as ferrous glycinate), 15 mg Zinc (as zinc arginate), 1 mg Copper (as copper lysinate), 2 mg Manganese (as manganese amino acid chelate), 120 mcg Chromium (as chromium amino acid chelate), 100 mcg Selenium (as selenomethionine), 500 mg Potassium (as potassium citrate), L-Glutamine, 200 mg Choline (as choline bitartrate), 5 g medium chain triglycerides, 500 mg bromelain and about 8 grams of a blend of the levorotatory form of the amino acids: leucine, lysine, threonine, tyrosine, histadine, isoleucine, valine and methionine.

5. A method for accelerating wound healing wherein the wound is the result of surgery performed on a patient comprising the step of orally administering a unit dose of the composition of claim 1 to said patient twice daily beginning at least two days prior to surgery and ending about 7 days after surgery.

6. A method of promoting wound healing in a patient following an elective surgical procedure comprising the step of orally administering a unit dose of a composition comprising about 10 grams of ornithine α-ketoglutarate to the patient twice daily beginning at least two days prior to said elective surgical procedure.

7. A method for accelerating wound healing in an athlete wherein said athlete engages in a seasonal sport activity and is at an elevated risk of injury while performing such sports activity, said method comprising the step of orally administering a unit dose of a composition comprising about 10 grams of ornithine α-ketoglutarate to the athlete twice daily beginning at least two weeks prior to engaging in said seasonal sports activity and daily thereafter until participation in said seasonal sports activity terminates.
